(19) United States
(12) Patent Application Publication
(10) Pub. No.: US 2014/0195174 A1
(43) Pub. Date: Jul. 10, 2014

(54) WELL WATER AND AQUIFER QUALITY MEASUREMENT AND ANALYSIS SYSTEM

(71) Applicants: David W. Machuga, Laurel, MD (US); John Francis Vontran, Perry Hall, MD (US); Matthew Thomas Poch, Dundalk, MD (US)

(72) Inventor: David W. Machuga, Laurel, MD (US)

(73) Assignees: David W. Machuga, Laurel, MD (US); John Francis Vontran, Perry Hall, MD (US); Matthew Thomas Poch, Dundalk, MD (US)

(21) Appl. No.: 14/147,014

(22) Filed: Jan. 3, 2014

Related U.S. Application Data
(60) Provisional application No. 61/750,388, filed on Jan. 9, 2013.

(51) Int. Cl. G01N 33/18

(52) U.S. Cl. CPC G01N 33/18
USPC 702/55

(57) ABSTRACT

Understanding the well water depth and aquifer quality is a challenging problem. With water demands increasing, a system that can both provide the local well user with critical information to protect their well and use this data to give a broader understanding of the water system is disclosed. The system provides four (4) basic functions: 1) to measure the level of water in one or more wells; 2) process the high resolution raw data to obtain a derived measure of well depth, draw down and refill times (transmissivity and storage coefficients); 3) transmit data to a local processing unit using existing wiring or wirelessly; and 4) transmit data to a shared processing unit or a network for local water reserves and broader data analysis on overall regional aquifer quality and trends for environmental impacts.

This application claims priority under 35 U.S.C. §119(e) from U.S. Provisional Application Ser. No. 61/750,388 entitled “Well Water and Aquifer Quality Measurement and Analysis System,” filed on Jan. 9, 2013, and which is incorporated herein by reference in its entirety.

### FIELD OF THE INVENTION

This invention relates generally to well water and aquifer quality, and more particularly to a system for measuring and analyzing the quality of well water and aquifers.

### BACKGROUND OF THE INVENTION

Water well and aquifer control and monitoring related developments pertain uniquely to the sensors for measuring the well or aquifer's level. The focus has been to provide well water depth data. Certain prior patents, such as U.S. Pat. No. 5,105,662 [Marsh, et al.], U.S. Pat. No. 5,207,251 [Cooks], U.S. Pat. No. 5,901,603 [Fiedler], and U.S. Pat. No. 6,490,919 [Bilinski et al.], detail sensors and methods for measuring water depth. Many new sensors are commercially available with direct measurement techniques using submerged ceramic sensors, rendering the measurement a state of the market technology.

These referenced prior sensors simply measure the depth of the well water, or head, and provide that to the user. While this data is useful, other parameters may provide greater insight into the overall well and surrounding aquifer quality. Understanding the overall aquifer quality allows the local user as well as regional interests to protect and extend the environmental water balance.

There are two (2) characteristics that reflect the quality of an aquifer for water production: transmissivity and storage coefficient. The transmissivity indicates how easily water can move through a geological formation and is defined as the rate at which water is transmitted through a unit width of an aquifer under a unit hydraulic gradient. The storage coefficient of an aquifer indicates the volume of water that can be removed from storage. This is defined as the volume of water an aquifer releases from storage per unit change in head per unit surface area of the aquifer. Without measurement of these characteristics, it is not possible to determine the overall health of the well and the supporting aquifer.

Drilling test wells and measuring the draw down over time in all of the wells during extended pumping tests that can last for 24 or more hours is typically how these quantities are determined. [Suggested Operating Procedures for Aquifer Pumping Tests, P.S. Osborne, United States Environmental Protection Agency, EPA/504/S-93/503, February 1993]

### SUMMARY OF THE INVENTION

It is one object of the present invention to provide a system for monitoring water well status and controlling water well usage. In one preferred embodiment, the system comprises a sensor configured to collect well data, a well data processing unit communicatively connected to the sensor and configured to receive well data from the sensor and transmit the well data to a processing unit; and a local or shared data processing unit communicatively connected with at least one of said well data processing unit and sensor and configured to receive said well data.

It is a further object of the present invention to provide a method for monitoring and controlling water wells. In one preferred embodiment, the method for monitoring and controlling water wells comprises the following steps: 1) collecting well data from a sensor; 2) transmitting the well data to at least one of a well data processing unit, a local processing unit, or a shared data processing unit; 3) determining well status based on the well data collected from the sensor; and 4) providing the well status data to a user.

### BRIEF DESCRIPTION OF THE DRAWINGS

FIG. 1 illustrates a system for monitoring and controlling water wells.

FIG. 2 is a schematic representation of a local processing unit.

FIG. 3 is a block diagram of a method implemented by the system described.

### DETAILED DESCRIPTION OF THE PREFERRED EMBODIMENTS

The details of one or more implementations may be better understood by referring to the following description, claims, and accompanying drawings in which corresponding call numbers relate to corresponding parts and elements of embodiments of the invention. The following description is of a particular embodiment of the invention, set out to enable one to practice an implementation of the invention, and is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Understanding the well water depth and aquifer quality is critical as increasing population places ever-higher demands on ground water. There is currently no integrated system that provides the local user with data that can be used to adjust pumping intervals and schedules. There is also a need for regional users to have such information for population center planning and drought impact analysis. The system allows users to protect their wells and have a broader understanding of the water system based on the data collected at individual wells and, in some preferred embodiments, later aggregated. The system described herein has four (4) basic functions: 1) to measure the level of water in a well; 2) process the high resolution raw data to obtain a derived measure of well depth, draw down and refill times (transmissivity and storage coefficient); 3) transmit data to a processing unit at the local house or other infrastructure using existing wiring or wirelessly; and 4) transmit data to a shared processing unit, such as a data center/web service, or a network for local water reserves and broader data analysis on overall regional aquifer quality and trends for environmental impacts.

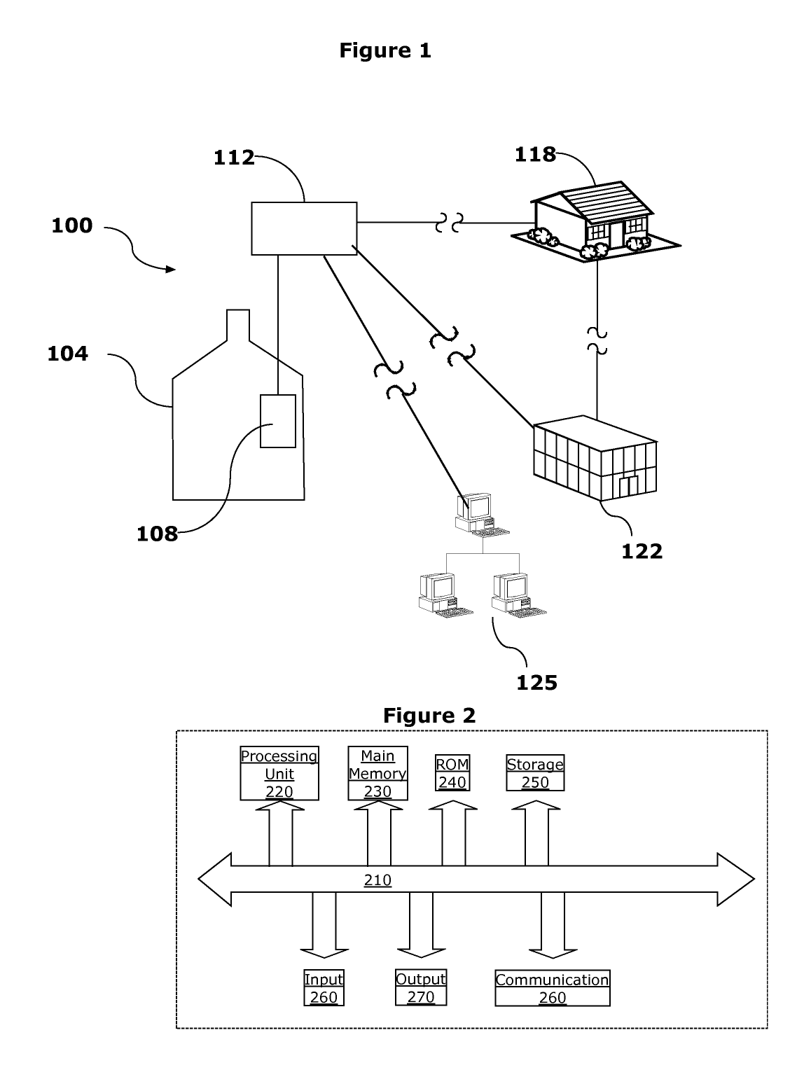

In accordance with the methods described herein, a well monitoring and control system is provided in order to take high-resolution measurement of well depth, calculate derived parameters of transmissivity and storage coefficient, transmit this back to a local processing unit at a house or other local infrastructure, and aggregate data at a data center on a shared processing unit or network. As shown in FIG. 1, the system 100 can be divided into three primary functional areas or components. These are: 1) a sensor 108, such as a submerged, in-well sensor at depth in a well 104; 2) a well data processing unit 112, such as a sealed electronics box in a wellhead; and 3) a local processing unit 118, comprising electronics at a house or other local infrastructure. In some embodiments, that local processing unit 118 is further connected to 4) a shared processing unit 122 and/or a network 125, which can be used to aggregate information from multiple wells, which allows for monitoring and controlling a number of wells simultaneously. In other embodiments, the well data processing unit 112 may be directly connected to the shared processing unit 122 or the network 125.

A “processing unit,” as utilized herein, identifies a hardware component that may include a processor, microprocessor, or processing logic that may interpret and execute instructions (e.g., executable code, software, computer programs, etc.). The processing unit may also include various processors for managing one or more functions. For example, the processing unit may include a processor for managing inputs and outputs. The processing unit may also include processors to perform floating point mathematical operations. In some embodiments, the processing unit may include a special-purpose microprocessor configured for fast execution of signal processing algorithms. If necessary, the processing unit may include additional processors subordinate to a main processor (back-end processors). In other embodiments additional processors may be used, such as controllers for multiple processors. It is understood that these processors may be integrated or separate and discrete processors.

The first component of the system 100 is the sensor 108. It is contemplated that a submerged, in-well sensor at depth is a preferred type of sensor 108; however, a person of ordinary skill in the art will recognize that any sensor 108 capable of collecting the data necessary for measuring transmissivity and storage coefficient of the well 104 can be used. The sensor may be a state of the market sealed pressure sensor that is secured to well 104 structure (i.e., the torque arrestor) near the pump. In some embodiments, the sensor 104 is based on a ceramic sensor head and electronics to allow pressure to be measured either by digital means or by direct voltage that is converted to digital data.

In alternative embodiments, the pressure sensor may be integrated with the pump housing. The sensor 108 is communicatively connected with the well data processing unit 112. In some embodiments, the sensor 108 is connected through an imbedded and sealed cable leading to the well data processing unit 112 located at the wellhead. In other embodiments, the sensor 108 is connected wirelessly to the well data processing unit 112.

As utilized herein, the term “communicatively connected” means connecting a particular element with another element by means that allow communication between the two, such as cables, wireless connections, fiber-optics and any other such means known by a person of ordinary skill in the art. The sensor 104 of the present system 100 measures well depth at sufficiently high resolution during pumping and recovery to determine the transmissivity and storage coefficient.

The sensor 108 is communicatively connected with the well data processing unit 112. In a preferred embodiment, the well data processing unit 112 is housed in a hermetically sealed housing for electronics with connectors providing an environmental seal against humidity, dust, dirt, rodents, and water. In a preferred embodiment, the well data processing unit 112 is configured to perform the following functions: i) sensor signal conditioning, ii) sensor signal digitization, iii) signal processing, iv) communication, v) power/power management, and vi) health and status monitoring. Each function of the well data processing unit 112 is described below.

The first function of the well data processing unit 112 is sensor signal conditioning. Conditioning is determined by the type of sensor implemented. Signal conditioning may include input signal buffering, DC offset removal, AC noise removal/filtering, signal amplification, and any other functions as required, as this list is not intended to be inclusive of all possible required functions.

The second function of the well data processing unit 112 is sensor signal digitization. If the sensor 108 is analog in nature (i.e., no built-in digital converter), an analog to digital converter is required for signal digitization. This function converts the analog signal to a digital representation for use in the digital processor. The final output of the sensor signal digitization is the input to the signal processing function.

The third function of the well data processing unit 112 is signal processing. The data collected from the sensor 108 is used to calculate the well's transmissivity and storage coefficients. The system 100 facilitates calculation of transmissivity and the storage coefficient by means of mathematical models describing groundwater flow towards the well based on high-resolution time measurements made by the sensor 108. Without measurement of these characteristics, it is not possible to determine the overall health of the well and the supporting aquifer.

The signal processing function of the well data processing unit 112 calculates the drawdown for the pumping period (and the volume pumped), and then determines the time required for full recovery to the pre-pump levels. The calculations can be based on mass balance approach and leverage of the Thiem (1906), Theis (1935) and Jacob (1946) equations. The general Jacob method can be used to determine the transmissivity of the aquifer using direct measurements of the head depth from the pumped well. More particularly, based on the change of head depth over time, the rate at which water is transmitted through a unit of width of the subject aquifer under a unit hydraulic gradient may readily be calculated. It is contemplated that such signal processing function may also be carried out at the shared processing unit in some preferred embodiments.

The storage coefficient (i.e., the volume of water the subject aquifer releases from storage per unit change in head depth per unit surface area of the aquifer) is determined as described above and based upon the change of depth in the well over time. The storage coefficient can also be estimated during drawdown; however, the value is not reliable using a single measurement. In one embodiment, the storage coefficient is determined during multiple pump cycles and the system takes into account the recovery time for the pumping cycles to refine the storage coefficient estimate using the determined transmissivity. For example, with a high transmissivity coefficient and low storage coefficient, the recovery will be quick initially. However, recovery will slow down before reaching the initial levels prior to pumping due to the demand from storage. In a further embodiment, as described in more detail below, the estimates can be further refined through communication with nearby wells. The output is not only a measurement of well depth (head), but also aquifer quality. This function is preferably implemented in a microcontroller that may also perform other functions. In an alternative embodiment, however, the measurements can be transmitted to other components of the system for further processing and for determination of the transmissivity and storage coefficients.

A fourth function of the well data processing unit 112 is communication. In a preferred embodiment, the well data processing unit 112 is designed and configured to eliminate the need for a user to go to the well in order to collect data from the well. In such embodiments, the well data processing unit is communicatively connected to a local processing unit 118, to a shared data processing unit 122, or to a network 125. It is contemplated that the well data processing unit is equipped with the required equipment for wired or wireless communications including power supply, hardware, antennas and other such elements as recognized by a person of ordinary skill in the art. The well data processing unit 112 may transmit well information to the local processing unit 118 using existing wiring or wireless technology for local display and controlling of the pump system to protect the well and aquifer. A person of ordinary skill in the art will recognize that such communicative connections and associated standard protocols include Ethernet (including standard IEEE 802 standards), Ethernet over power, Wi-Fi, cellular (i.e. 3G, 4G, 4G LTE, etc. . . . ), digital subscriber line (“DSL”), asynchronous digital subscriber line (“ADSL”), asynchronous transfer mode (“ATM”), integrated digital services network (“IDSN”), personal communications services (“PCS”), transmission control protocol/Internet protocol (“TCP/IP”), serial line Internet protocol/point to point protocol (“SLIP/PPP”), satellite links, and other links as appropriate and available as this list is not intended to be inclusive of all possible communication links. In the case of Ethernet over power, a module will convert all data from the electronics to a data connection over the in-place power lines for pump power. This allows for data to get back to the house or central infrastructure for display and long-haul wide-area communications without the need for additional wiring to the well.

A further functionality of the well data processing unit 112 provides for power management of the system. Power may be obtained from multiple potential sources. These include solar, battery, direct connection, power leaching from the pump system, and any other means applicable from the local installation as this list is not intended to be inclusive of all possible available power connections. In the case of power leaching, internal batteries would preferably be recharged each time the well pump is operated. This alleviates the need for additional wiring from the local infrastructure. In this case, the health of the local battery would also be monitored.

A further function of the well data processing unit 112 provides health and status monitoring of one or more components of the system. This functionality of the well data processing unit 112 also allows, through the communication functionality, users to evaluate the status of one or more components of the system. This may include pressure sensor operation, system and environment temperature, humidity, power status, and any other parameters deemed appropriate as this list is not intended to be inclusive of all possible monitored parameters.

In certain implementations, if this system is installed with a new well allowing for separate wiring, the sealed processing box, i.e., well data processing unit 112, can be combined and placed with the local processing unit 118, below, in the house or local infrastructure.

A third component of the system is a local processing unit 118. The local processing unit 118 can be located at an individual house served by a particular well 104. In other embodiments that local processing unit 118 is located at a point within a community that utilizes a single well 104. The local processing unit 118 may comprise a personal computer (“PC”), a cellular phone, a mobile phone, a tablet computer, or any other type of device that can be communicatively connected to the sensor 108, as explained above. As shown in FIG. 2, the local processing unit 118 may comprise a bus 210, a processing unit 220, a main memory 230, a read only memory (ROM) 240, a storage device 250, an input device 260, an output device 270, and a communication interface 280.

Main memory 230, as utilized herein, refers to dynamic storage devices that may store information and instructions for execution by the processing unit (e.g., such as Random Access Memory (RAM). ROM 240 refers to static storage devices that may store static information and instructions for use by a processing unit. A storage device 250 is any type of device that can be used for data storage other than that stored in ROM 240 or main memory 230, and may include magnetic, optical, removable devices, USB enabled devices, or other recording media recognized by a person of ordinary skill in the art. A person of ordinary skill in the art would also recognize that input 260 and output 270 devices are any devices that can be utilized to allow the user to interact with the system, such as keyboards, screens, touchscreens, pens/stylus, voice recognition devices, a mouse, touchpads, and other related mechanisms.

The local processing unit 118 is configured to perform several functions: i) communications, ii) local storage, iii) well pump control, and iv) local data display. In some preferred embodiments, the local processing unit may also be configured to process well data to determine well status, including transmissivity and storage coefficient.

The communication interface 280 facilitates communication between the local processing unit 118 and the sensor 108 or the well processing unit 112. It allows for receiving local data transmissions from the electronics box of the well processing unit 112 at the wellhead. It is contemplated that the communication interface 280 may also allow the user to interact with the network 125 or the shared data processing unit 122. The local storage of data function allows the local processing unit 118 to store aquifer or well data. This data provides a local archive of historical data that can be utilized to determine or monitor long term trending. Such storage function is enabled through the storage device 250. The communication interface 280 may also be configured to serve as a wide-area transmitter that provides the connection to upload the well and aquifer data to the shared data processing unit 122, which may be a data center, using electronic means such as XML web services, etc. The transmission of this data is for local ownership Internet viewing and regional aggregation. The types of communication can include modem, DSL, cellular (i.e. 3G, 4G, 4G LTE, etc . . . ), satellite links, and such other connections as may be appropriate and available as this list is not intended to be inclusive of all possible available communication links.

Another function of the local processing unit 118 is to allow the user to control the draw of water from the well. Through the communication interface 280, the user may turn the well pump on or off based on the data provided by the system. If the well properties are changing (i.e., well not refilling as quickly as previously), rather than have the well continuously pump to fill the reservoir, separate pumping cycles can be implemented to allow for the aquifer to refill the well. This zero-cost change prevents the well from pumping dry, protecting the pump equipment. In addition, notification of this status to the well owner allows for scheduling for drilling of a deeper well. In some embodiments, the local processing unit may be programed to take specific actions in regards to well usage based on well status, including the transmissivity and storage coefficient.

One advantage of the present system is the ability for a user to assess well depth (head) and aquifer quality through local display functionality. The output device 270 provides the user with a local understanding of the well quality. This data is presented in the house or local infrastructure to alleviate the need to check for data at the individual wellheads. If the system 100 is part of a new installation with an integrated pump sensor, the second functional area electronics may be co-located with this functional area in the house or local infrastructure since the new install would require new power wiring to the well. In addition to local data display, the data is transmitted to a data analysis center through available communication links for further processing.

In a further embodiment of the present system, a shared data processing unit 122 receives data input from the local processing unit 118 or the well data processing unit 112. In some embodiments, the shared data processing unit 122 is a data center. In other embodiments, the shared data processing unit includes a web page generator for an individual subscriber to check current data and historical data. The shared data processing unit 122 may also provide the individual well user data derived from nearby wells that can be used as “test wells” when pumping occurs. Before the development of this system 100, a holistic view of ground water was not available. The shared data processing unit 122 data center's primary function is to serve as a data aggregator for consumption by local, state, and federal governments, or any other entity charged with water management for an aquifer, for environmental monitoring by providing an overall view of groundwater changes at the regional level. It is contemplated that the data may be aggregated anonymously to protect privacy.

The data collected from the well and provided to the local processing unit 118 at the house or local infrastructure is received and archived on local storage at the shared data processing unit 122. This data can be used to control the pumping rates and times of the well system.

The system can be implemented in multiple wells, which can send data back to a central infrastructure for a single, aggregated local analysis. The data received at the shared data processing unit 122 can be aggregated from multiple sources. This data can be sent by simple XML messages over the Internet, or as short text messages over the cellular network or through any other communication means described here. Sources that are close to one another can be treated as test wells relative to traditional drawdown tests to verify correct calculation of transmissivity and storage coefficients. In addition, aggregated data can be used to show broad areas of aquifer depth and quality, providing regional planners and governments previously unavailable data. This data can further be sent back to the individual well operators to further refine the collection, processing, and pump operation parameters.

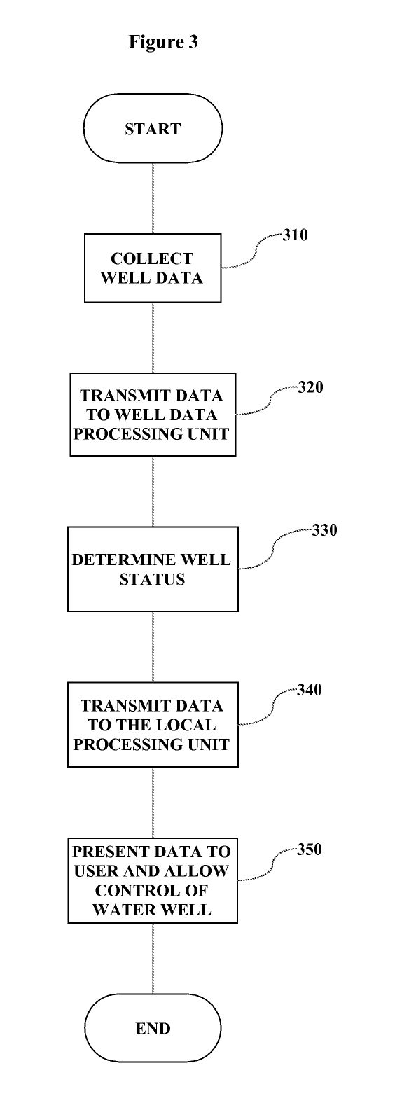

The system described above can be used to implement the method described in FIG. 3. In a first step 310, the pressure sensor measures or collects well data. In a second step 320, the sensor transmits the data to the well data processing unit. The well data collected by the sensor is conditioned and digitized. The conditioning step includes input signal buffering, DC offset removal, AC noise removal/filtering, and signal amplification. If necessary, analog signals are then converted to digital signals for further processing. In a third step 330, the well status is determined. Well status includes the well's transmissivity and storage coefficients calculated by the well data processing unit based on the data received from the sensor. In a fourth step 340 the data is transmitted to the local processing unit 118. It is contemplated that the information is displayed to a user through the local processing unit 118 and the user is given the ability to control use of the well. In the final step 350, the user interface presents the data to a user and allows the user to control the amount of water drawn from the well.

The method described above is implemented in the system through a software program stored in a computer readable media. The software program comprises a set of instructions that are read and implemented by a processing units described above. As used in this description, the term “computer readable medium” or “computer readable media” refer to any device, implement, or mechanism, used to store or provide executable instructions (e.g., software and computer programs) to the various components of the system for execution by a processing unit. Examples of such components include the well data processing unit 112, the local processing unit 118, and the shared data processing unit 122. These computer readable media are means for providing executable code, programming instructions, and software to the various components of the system. The computer readable media are also means to store such executable code, programming instructions, and software such that the processing units in the system can access the particular set of instructions and implement the method described above in the system.

The invention may be embodied in other specific forms without departing from the spirit or essential attributes thereof and, accordingly, the described embodiments are to be considered in all respects as being illustrative and not restrictive, with the scope of the invention being indicated by the appended claims, rather than the forgoing detailed description, as indicating the scope of the invention as well as all modification which may fall within a range of equivalency which are also intended to be embraced therein.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. A system for monitoring water well status and controlling water well usage, comprising:
a sensor configured to collect well data,
a well data processing unit configured to calculate the well's storage coefficient and transmissivity; wherein said well data processing unit is communicatively connected to said sensor and configured to receive well data from said sensor and transmit said data; and
a local or shared data processing unit communicatively connected with at least one of said well data processing unit and sensor and configured to receive said well data.

2. The system of claim 1, wherein at least one of said data processing units is further configured to limit usage of the well based on the well's transmissivity and storage coefficient.

3. The system of claim 2, wherein at least one of said data processing units is further configured to allow a user to control usage of the well.

4. A method for monitoring and controlling water wells, comprising:
collecting well data from a sensor;
transmitting the well data to at least one of a well data processing unit, a local processing unit, or a shared data processing unit;
determining the well storage coefficient and transmissivity based on the well data collected from the sensor; and
providing the well status data to a user.

5. The method of claim 4, further comprising enabling a user to control well usage based on the well's storage coefficient and transmissivity.

6. The method of claim 4, further comprising limiting well usage based on storage coefficient and transmissivity.

7. A system for monitoring multiple water wells and controlling water well usage, comprising:
at least two sensors configured to collect well data from at least two wells,
a well data processing unit configured to calculate the well's storage coefficient and transmissivity; wherein said well data processing unit is communicatively connected to said sensor and configured to receive well data from said sensor and transmit said data; and
a local or shared data processing unit communicatively connected with at least one of said well data processing unit and sensor and configured to receive said well data.

8. The system of claim 7, wherein at least one of the well data processing unit, local processing unit, and shared processing units is configured to calculate the each water well's status from said data.

9. The system of claim 7, wherein at least one of said data processing units is further configured to limit usage of at least one well based on the well's transmissivity and storage coefficient.

10. The system of claim 9, wherein at least one of said data processing units is further configured to allow a user to control usage of the well.
